(19) United States
(12) Patent Application Publication
(10) Pub. No.: US 2025/0352038 A1
(43) Pub. Date: Nov. 20, 2025

(54) SUCTION VALVE FOR AN ENDOSCOPE

(71) Applicant: Boston Scientific Scimed, Inc., Maple Grove, MN (US)

(72) Inventors: Jenny Dandin, Worcester, MA (US); John B. Golden, Norton, MA (US)

(73) Assignee: Boston Scientific Scimed, Inc., Maple Grove, MN (US)

(21) Appl. No.: 19/205,393

(22) Filed: May 12, 2025

Related U.S. Application Data
(60) Provisional application No. 63/647,287, filed on May 14, 2024.

(51) Int. Cl. A61B 1/00

(52) U.S. Cl. CPC A61B 1/00068

(57) ABSTRACT

Devices, systems, and methods for a suction valve assembly for a medical device. The valve assembly includes a valve cap and valve stem with a central lumen that fits within the internal valve well of a valve body. The central lumen opens in the bottom of the valve stem, where the valve body connects to a source of suction. The valve is lowered to open a working channel to the source of suction. At least one, and as many as three, seals surround the valve stem to obstruct fluid flow along the side of the valve well.

### CROSS-REFERENCE TO RELATED APPLICATIONS

This application claims the benefit of U.S. Provisional Patent Application Ser. No. 63/647,287 filed on May 14, 2024, the disclosure of which is incorporated herein by reference.

### TECHNICAL FIELD

This disclosure relates generally to valve assemblies and methods, and particularly to suction supply valve assemblies and methods for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a valve assembly for a medical device includes a valve body having an internal valve well; a valve cap; a valve stem connected to the valve cap and configured to translate within the valve well between an upper position and a lower position; and a first seal extending circumferentially around the valve stem. The valve body has a source passage and a working passage each connected to the internal valve well. The valve stem has two channel inlets each opening into a side surface of the valve stem and a central lumen extending from the air inlets in the valve stem an opening into a bottom surface of the valve stem. The first seal contacts an internal wall of the valve well to obstruct fluid flow between the working passage and the top of the valve well when the valve stem is in the upper position and when the valve stem is in the lower position.

Alternatively or additionally to any of the examples above, the valve assembly can further include a second seal extending circumferentially around the valve stem, the second seal contacting the internal wall of the valve well to obstruct fluid flow between the working passage and the top of the valve well when the valve stem is in the upper position, and to obstruct fluid flow between the working passage and the bottom of the valve well when the valve stem is in the lower position.

Alternatively or additionally to any of the examples above, the valve assembly can further include a third seal extending circumferentially around the valve stem, the third seal contacting the internal wall of the valve well to obstruct fluid flow between the working passage and the bottom of the valve well when the valve stem is in the upper position and when the valve stem is in the lower position.

Alternatively or additionally to any of the examples above, the first, second, and third seals can be formed from injection molding into grooves in the side surface of the valve stem.

Alternatively or additionally to any of the examples above, the first, second, and third seals can comprise a single seal component.

Alternatively or additionally to any of the examples above, grooves in the side surface of the valve stem can be joined by channels. The seal component comprising the first, second, and third seals can be formed from a single process of injection molding into the grooves and channels.

Alternatively or additionally to any of the examples above, the first seal can be a flexible seal formed of a material with a durometer less than the durometer of a material forming the valve stem.

Alternatively or additionally to any of the examples above, the second seal can be a flexible seal formed of a material with a durometer less than the durometer of a material forming the valve stem.

Alternatively or additionally to any of the examples above, the third seal can be a flexible seal formed of a material with a durometer less than the durometer of a material forming the valve stem.

Alternatively or additionally to any of the examples above, each of the first, second, and third seals can be formed of the same material using the same manufacturing process.

Alternatively or additionally to any of the examples above, the valve stem can be injection-molded thermoplastic.

Alternatively or additionally to any of the examples above, the valve assembly can further include

Alternatively or additionally to any of the examples above, the valve assembly can further include a valve collar configured to attach to the valve body, the valve collar remaining stationary relative to the valve body when the valve stem moves between the upper and lower positions; and a spring member connected to the valve collar and the valve cap, the spring member biased to move the valve cap upward to return the valve stem to the upper position from the lower position.

Alternatively or additionally to any of the examples above, the valve collar can include a plurality of latches sized and positioned to press against the valve body when the valve collar is attached to the valve body.

In another example, an endoscopic medical device comprises an endoscopic probe; and the valve assembly of any of the above examples coupled to the endoscope for use in an endoscopic procedure.

Alternatively or additionally to any of the examples above, the endoscopic medical device can further include an endoscopic operating handle including the valve body of the valve assembly.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3A depicts perspective and exploded views of an illustrative supply valve;

FIG. 3B depicts a perspective view of an illustrative valve stem and seals;

FIGS. 3C and 3D depict top and bottom perspective views of a valve collar;

FIG. 4A depicts a plan view of an illustrative valve body;

FIG. 4B depicts a schematic cross-sectional view of the valve body;

FIG. 5A depicts a schematic cross-sectional view of the illustrative supply valve and body, with the valve in a first configuration;

FIG. 5B depicts a schematic cross-sectional view of the illustrative supply valve and body, with the valve in a second configuration;

FIG. 6A depicts perspective views of an illustrative valve stem and seals;

FIGS. 6B and 6C depict schematic cross-sectional views of an illustrative supply valve and body, in first and second configurations; and

FIG. 7 depicts perspective views of an illustrative valve stem and seals.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

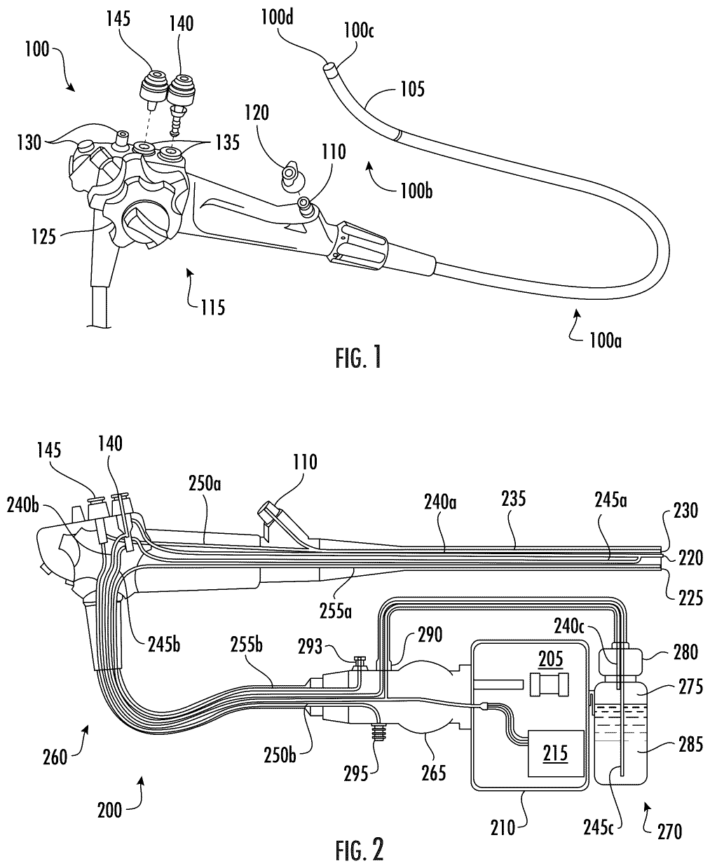

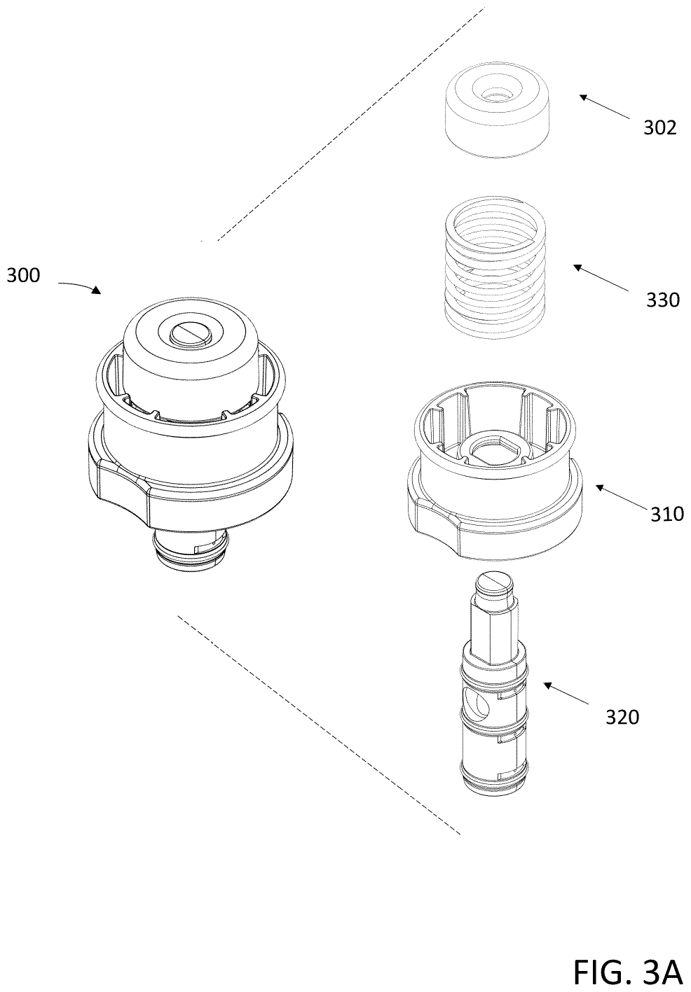

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serves as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

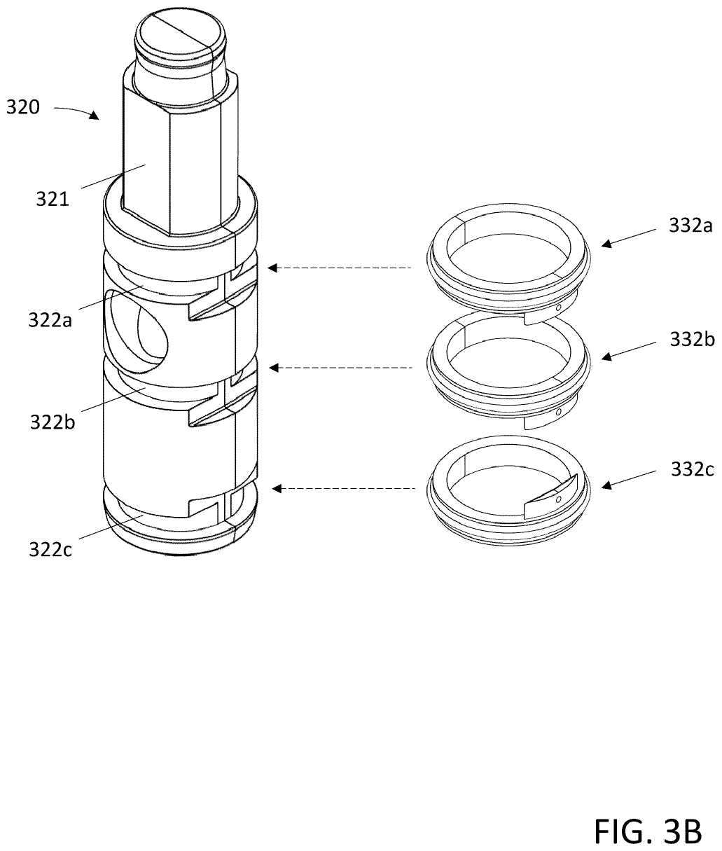

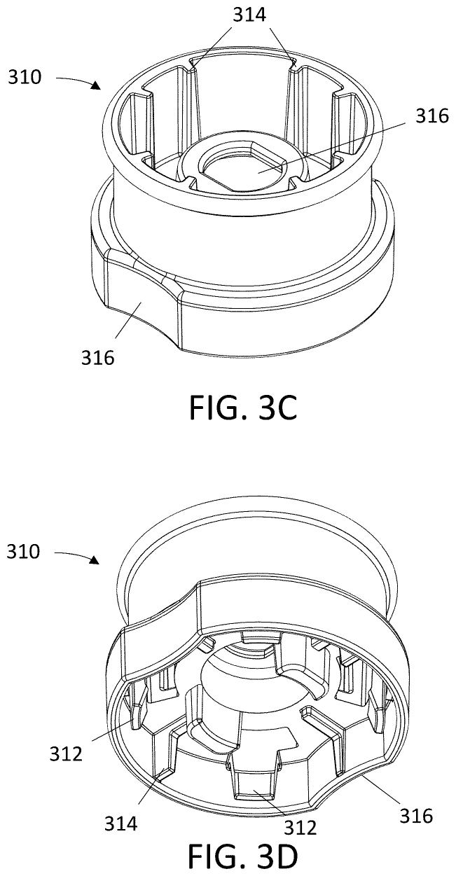

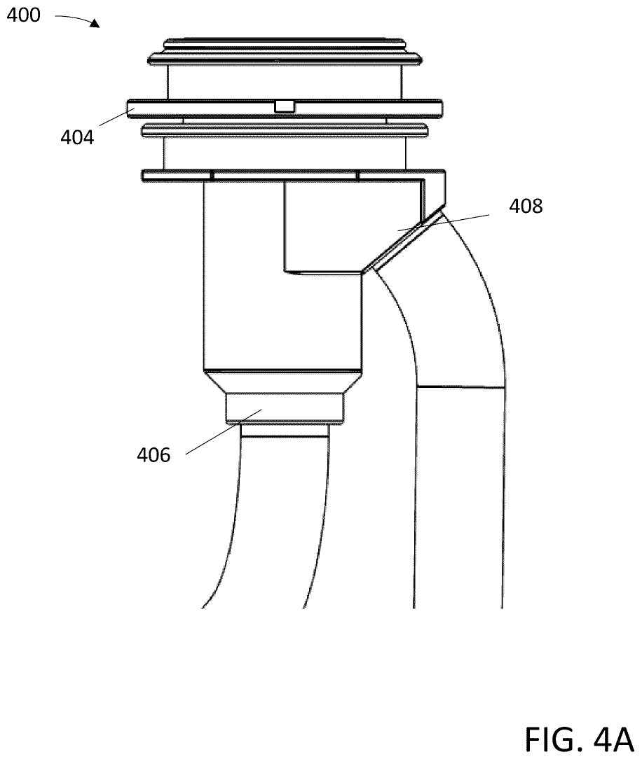

An example of a removable suction valve 300 is illustrated in FIGS. 3A-3D. The valve 300 includes a valve cap 302, a valve collar 310, and a valve stem 320. A spring member 330 connects the valve cap 302 to the valve collar 310, while seals 332a-c are disposed along the outside surface of the valve stem 320. To open the valve 300, a user presses downward on the valve cap 302, which moves the cap 302 and stem 320 relative to the collar 310. When the cap 302 is released, the spring member 330 returns the valve 300 to the default position.

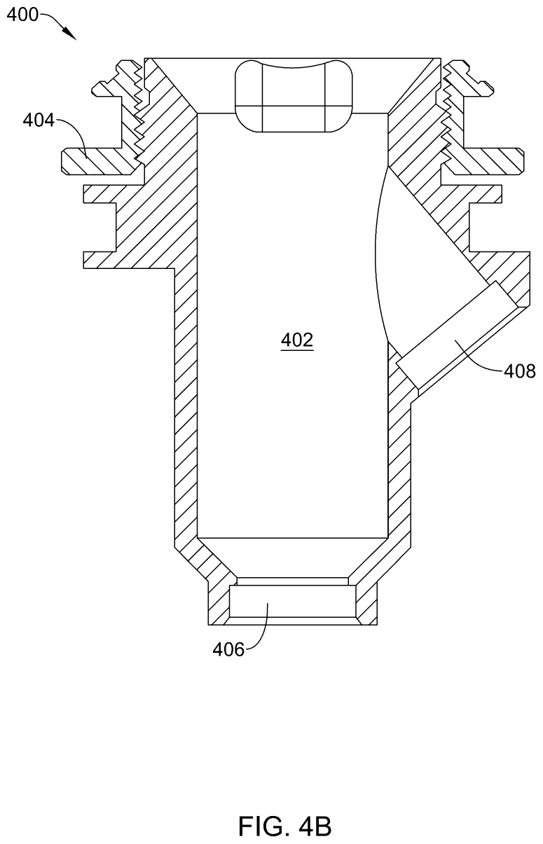

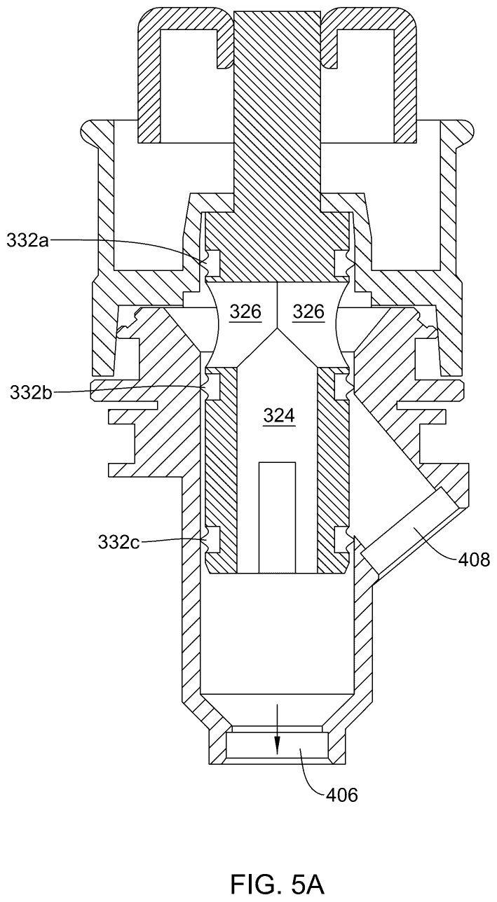

The valve 300 is inserted into a valve well, such as one of the locations 135 described above and illustrated in FIGS. 1 and 2. As shown in FIGS. 4A-4C, a valve body 400 includes an internal valve well 402 sized and shaped to receive the stem 320 of the valve 300, as well as alternative valve designs (including each of those illustrated and described below). The valve body 400 also includes a source passage 406 communicating with a source of suction as described above with respect to suction feed line 250b, and a working passage 408 communicating with the working channel 235.

The valve collar 310 attaches to the body 400 and remains stationary relative to the body 400 when the valve 300 is actuated. The cap 302, stem 320, and seals 332a-c move downward within the valve well 402 when the valve 300 is actuated. Actuating the valve 300 compresses the spring 330 between the cap 302 and the collar 310. The spring 330 presses upward to return the valve 300 to its upward position when it is no longer pressed down.

As shown in FIG. 3B, the valve stem 320 may be injection-molded plastic. The stem 320 may include a neck 321 with both flat and curved faces so that only two opposite rotational orientations are possible for the interface between the stem 320 and the collar 310. The outer surface of the stem 320 includes three grooves 322a-c to accommodate the seals 332a-c. In some embodiments, the seals 332a-c may be injection-molded plastic or silicon that is formed around the stem 320 within the grooves 322a-c. As shown, the grooves 322a-c may include cut-outs or other features to accommodate the injection molding.

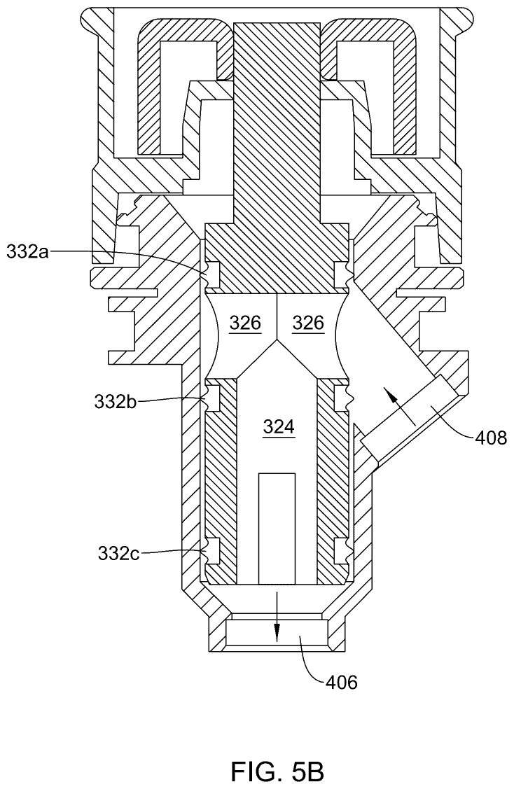

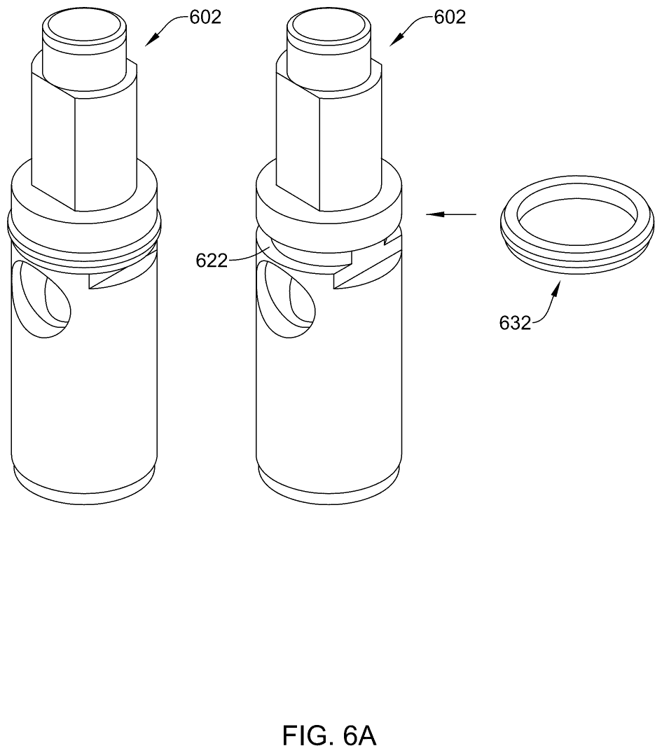

In some implementations, the rigid thermoplastic used to form the valve stem 320 may drive certain design elements for the shape and size of valve components. For example, the central lumen 324 of the valve stem 320 (shown in FIGS. 5A and 5B) may be slightly tapered to better accommodate injection molding. The structure and design of valve components may similarly be the result of the material choice and manufacturing process.

As shown in FIGS. 3C and 3D, the underside of the valve collar 310 includes a plurality of latches 312 spaced radially between a plurality of ribs 314. In some implementations, the latches 312 resiliently press against a lip of the endoscope to clip the collar 310, and consequently the valve 300, in place when installed. Indentations 316 on opposite sides of the collar accommodate an adjacent color for an additional valve, such as a gas/water valve placed in the valve location 135 as described above. The central hole 318 of the collar is shaped with two curved sides and two flat sides to accommodate the neck of the valve stem 320.

FIGS. 4A and 4B show the valve body 400, which as noted above is part of the endoscopic handle 115. A nut 404 is affixed along the outside surface of the body 400. The top of the valve well 402 is open to receive a valve, while the source passage 406 and working passage 408 connect to a source of suction and a working channel, respectively, for applying suction to an endoscope 100.

FIG. 5A shows a closed configuration for the valve 300 within the valve well 402. The valve stem 320 includes two air inlets 326 connected to a central lumen 324. When the stem 320 is in the upper position shown in FIG. 5A, the second seal 332b and third seal 332c flank the opening to the working passage 408. In this configuration, the source of suction is not in communication with the working channel.

FIG. 5B shows an open configuration in which the valve stem 320 is positioned lower in the body 400. Downward force on the valve cap 302 causes and maintains this position; when the valve cap 302 is released, the spring member 330 returns the valve 300 to its previous position. In this configuration, the upper seal 332a is above the opening to the working passage 408 to obstruct air flow from the top of the valve well 402. Air, fluid, and debris are permitted to pass through the working passage 408, through the internal channels 326 and 324, and into the source passage 406. Upon release of the downward force and return of the valve cap 302 to its previous position, the placement of the seals 332a and 332b again prevents the suction from being applied at the working passage 408.

The valve stem 320 may couple to the cap 302 in any suitable manner. In some cases, a portion (e.g., a proximal portion) of the valve stem 320 extending proximally of a hole in the cap 302 may be coupled to the cap 302 via one or more suitable coupling mechanisms. Example suitable coupling mechanisms include, but are not limited to, adhesives, a threaded connection, a luer lock connection, a snap connection, a ball-detent connector, a friction fit, and/or additional or alternative coupling mechanisms. The valve stem 320 may have any suitable configuration configured to adjust positions within the valve well, adjust flow paths to the air and water supplies and feeds, and couple to the cap 302.

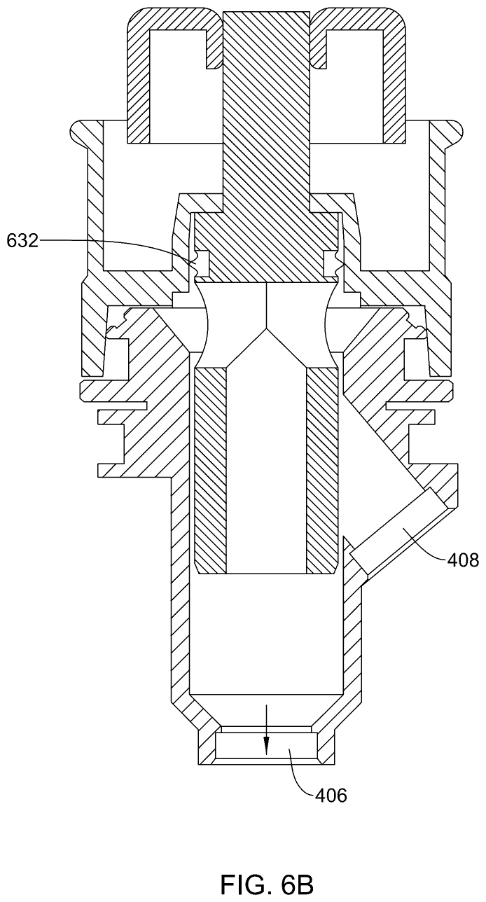

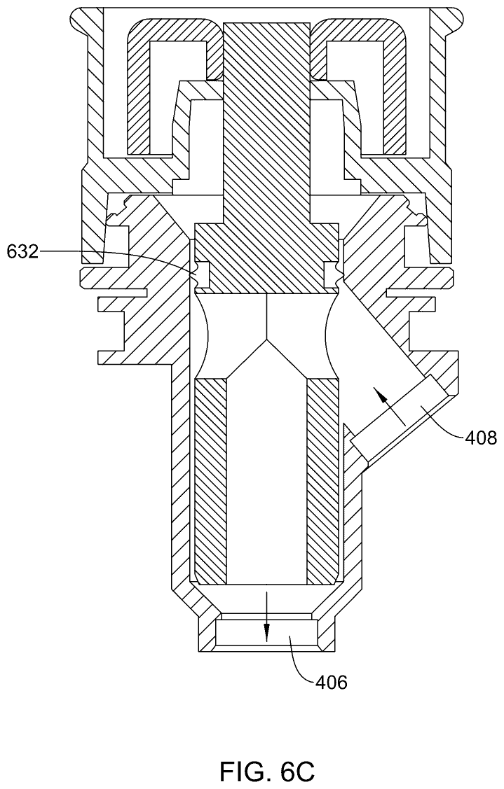

An alternative embodiment of a valve stem 620 is illustrated in FIGS. 6A-6C. In place of the three seals 332a-c described above, the valve stem 620 includes only a single seal 632. Here, the interface between the outer surface of the valve stem 620 and the inner surface of the valve well 402 acts to obstruct fluid flow in the absence of lower seals. One of ordinary skill will recognize that the absence of the two lower seals may save manufacturing costs but may require greater precision in matching the outer diameter of the valve stem 620 to the inner diameter of the valve well 402.

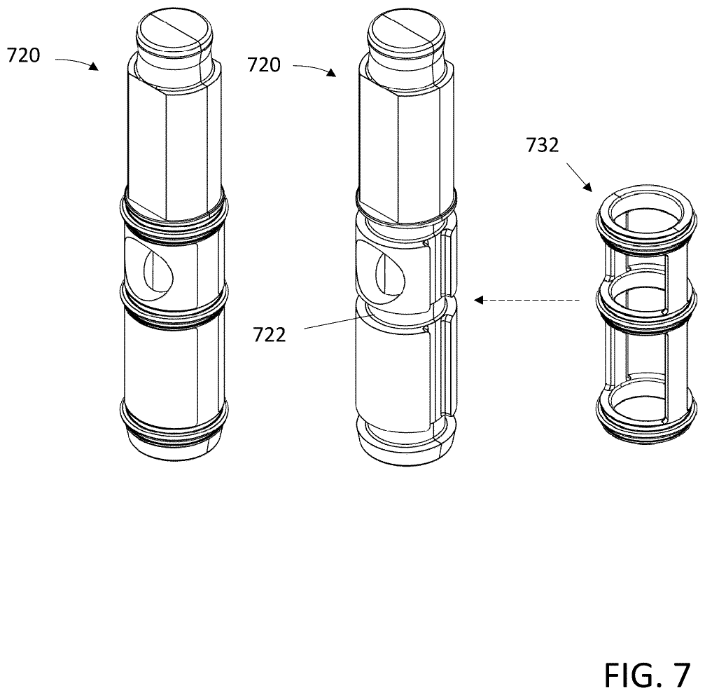

A further embodiment of a valve stem 720 is shown in FIG. 7. Here, three seals are formed as a single molded component 732. The external groove 722 comprises three ring grooves with channels between them for injection molding of the entire seal component 732 as one piece. Use of the side channels to connect the ring grooves may allow for the injection molding to occur more rapidly and use fewer injection points when forming the seal component 732.

The valve 300 may be formed in any suitable manner. In some cases, though not required, the valve 300 may be formed using a molding process, an injection molding process, a casting process, a finishing process, sanding, and/or by or with one or more additional or alternative manufacturing techniques. In one illustrative example, the valve 300 may be formed using an injection molding process.

The valve stem 320, 620, or 720 and the seals 332a-c, 632, or 732 may be formed from any suitable materials. In some cases, the valve stem 320, 620, or 720 may be formed from a first material and the seals 332a-c, 632, or 732 may be formed from a second material, where the second material may be the same as or different than the first material. The valve stem 320, 620, or 720 may be formed from a hard or rigid polymer and the seals 332a-c, 632, or 732 may be formed from a flexible polymer, but this is not required. In one example, the valve stem 320, 620, or 720 may be formed from one or more of polymer, acrylonitrile butadiene styrene (ABS), polycarbonate, and/or other suitable materials. In another example, the seals 332a-c, 632, or 732 may be formed from one or more of a polymer, thermoplastic elastomers (TPE), thermoplastic polyurethane (TPU), liquid silicone rubber (LSR), and/or other suitable materials.

The material of the seals 332a-c, 632, or 732 may have any suitable durometer. In one example, the material of the seals 332a-c, 632, or 732 when formed on the valve stem 320, 620, or 720 may have a durometer in a range of about 20-80 shore A, about 30-60 shore A, and/or other suitable values within one or more other suitable ranges of durometer, but could be softer or firmer depending on the geometry used for the seals and the amount of interference desired with the inner wall of the valve well 402. In one example, the seals 332a-c, 632, or 732 may be formed from silicone with a durometer in a range of 40-50 shore A, but this is not required.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A valve assembly for a medical device, comprising:
a valve body having an internal valve well, the valve body having a source passage and a working passage each connected to the internal valve well;
a valve cap;
a valve stem connected to the valve cap and configured to translate within the valve well between an upper position and a lower position, the valve stem comprising:
two channel inlets each opening into a side surface of the valve stem, and
a central lumen extending from the air inlets in the valve stem an opening into a bottom surface of the valve stem; and
a first seal extending circumferentially around the valve stem, the first seal contacting an internal wall of the valve well to obstruct fluid flow between the working passage and the top of the valve well when the valve stem is in the upper position and when the valve stem is in the lower position.

2. The valve assembly of claim 1, further comprising a second seal extending circumferentially around the valve stem, the second seal contacting the internal wall of the valve well to obstruct fluid flow between the working passage and the top of the valve well when the valve stem is in the upper position, and to obstruct fluid flow between the working passage and the bottom of the valve well when the valve stem is in the lower position.

3. The valve assembly of claim 1, further comprising a third seal extending circumferentially around the valve stem, the third seal contacting the internal wall of the valve well to obstruct fluid flow between the working passage and the bottom of the valve well when the valve stem is in the upper position and when the valve stem is in the lower position.

4. The valve assembly of claim 3, wherein the first seal, the second seal, and the third seal are formed from injection molding into grooves in the side surface of the valve stem.

5. The valve assembly of claim 3, wherein the first seal, the second seal, and the third seal comprise a single seal component.

6. The valve assembly of claim 5, wherein grooves in the side surface of the valve stem are joined by channels, and wherein the seal component comprising the first seal, the second seal, and the third seal are formed from a single process of injection molding into the grooves and channels.

7. The valve assembly of claim 1, wherein the first seal is a flexible seal formed of a material with a durometer less than the durometer of a material forming the valve stem.

8. The valve assembly of claim 2, wherein the second seal is a flexible seal formed of a material with a durometer less than the durometer of a material forming the valve stem.

9. The valve assembly of claim 3, wherein the third seal is a flexible seal formed of a material with a durometer less than the durometer of a material forming the valve stem.

10. The valve assembly of claim 9, wherein each of the first seal, the second seal, and the third seal are formed of the same material using the same manufacturing process.

11. The valve assembly of claim 1, wherein the valve stem is injection-molded thermoplastic.

12. The valve assembly of claim 1, further comprising:
a valve collar configured to attach to the valve body, the valve collar remaining stationary relative to the valve body when the valve stem moves between the upper and lower positions; and
a spring member connected to the valve collar and the valve cap, the spring member biased to move the valve cap upward to return the valve stem to the upper position from the lower position.

13. The valve assembly of claim 12, wherein the valve collar comprises a plurality of latches sized and positioned to press against the valve body when the valve collar is attached to the valve body.

14. An endoscopic medical device, comprising:
an endoscopic probe; and
a valve assembly coupled to the endoscope, the valve assembly comprising:
a valve body having an internal valve well, the valve body having a source passage and a working passage each connected to the internal valve well;
a valve cap;
a valve stem connected to the valve cap and configured to translate within the valve well between an upper position and a lower position, the valve stem comprising:
two channel inlets each opening into a side surface of the valve stem, and
a central lumen extending from the air inlets in the valve stem an opening into a bottom surface of the valve stem; and
a first seal extending circumferentially around the valve stem, the first seal contacting an internal wall of the valve well to obstruct fluid flow between the working passage and the top of the valve well when the valve stem is in the upper position and when the valve stem is in the lower position.

15. The endoscopic medical device of claim 14, further comprising an endoscopic operating handle including the valve body of the valve assembly.

16. The endoscopic medical device of claim 14, the valve assembly further comprising a second seal extending circumferentially around the valve stem, the second seal contacting the internal wall of the valve well to obstruct fluid flow between the working passage and the top of the valve well when the valve stem is in the upper position, and to obstruct fluid flow between the working passage and the bottom of the valve well when the valve stem is in the lower position.

17. The endoscopic medical device of claim 16, the valve assembly further comprising a third seal extending circumferentially around the valve stem, the third seal contacting the internal wall of the valve well to obstruct fluid flow between the working passage and the bottom of the valve well when the valve stem is in the upper position and when the valve stem is in the lower position.

18. The endoscopic medical device of claim 17, wherein the first seal, the second seal, and the third seal of the valve assembly are formed from injection molding into grooves in the side surface of the valve stem.

19. The endoscopic medical device of claim 14, the valve assembly further comprising:
a valve collar configured to attach to the valve body, the valve collar remaining stationary relative to the valve body when the valve stem moves between the upper and lower positions; and
a spring member connected to the valve collar and the valve cap, the spring member biased to move the valve cap upward to return the valve stem to the upper position from the lower position.

20. The endoscopic medical device of claim 19, wherein the valve collar comprises a plurality of latches sized and positioned to press against the valve body when the valve collar is attached to the valve body.
